(19) United States
(12) Patent Application Publication
(10) Pub. No.: US 2014/0073557 A1
(43) Pub. Date: Mar. 13, 2014

(54) FGFRI-BASED ANTAGONISTS WITH IMPROVED GLYCOSAMINOGLYCAN AFFINITY AND METHODS OF USING SAME

(71) Applicants: Andreas Kungl, Graz (AT); Martina Theuer, Graz (AT)

(72) Inventors: Andreas Kungl, Graz (AT); Martina Theuer, Graz (AT)

(73) Assignee: PROTAFFIN BIOTECHNOLOGIE AG, Graz (AT)

(21) Appl. No.: 14/005,523

(22) PCT Filed: Mar. 16, 2012
(86) PCT No.: PCT/EP2012/054723

§ 371 Date: Nov. 18, 2013

(30) Foreign Application Priority Data
Mar. 16, 2011 (EP) 11158490.0
Jul. 5, 2011 (EP) 11172689.9

(51) Int. Cl. C07K 14/71

(52) U.S. Cl. CPC C07K 14/71
USPC 514/1.9; 530/350; 536/23.5; 435/320.1; 435/364; 435/361; 435/252.33; 514/9.1

(57) ABSTRACT

A novel approach for inhibiting FGF2/FGFR1-mediated signalling is presented which is based on FGFR1 mutations to introduce higher affinity for the natural GAG co-receptors into the soluble part of the FGF1 receptor, preferably into the D2/D3 domains. Such recombinant drugs are expected to disrupt the natural FGF2/FGFR1/GAG triple complex by competing with the wtFGFR1 for GAG binding

The present invention relates to novel mutants of fibroblast growth factor receptor 1 (FGFR1) which preferably constitute only the soluble part, preferably the D2 or D2/D3 domain of the receptor and which exhibit increased glycosaminoglycan (GAG) binding affinity, and to their use for therapeutic treatment of diseases, preferably of cancer.

### BACKGROUND OF THE INVENTION

Angiogenesis is defined as a vascular neoformation usually of capillary origin. This phenomenon is important during development and under several physiological and/or pathological conditions. Angiogenic growth factors are currently targets of intense efforts to inhibit deregulated blood vessel formation in diseases such as cancer.

An important angiogenic molecule is the basic fibroblast growth factor (bFGF), also known as FGF2, which is the prototype of the FGF family. Studies have correlated the presence of FGFs and their receptors with different types of cancers and with a great tendency to metastasis (Girl et al., Clin Cancer Res. [1999], 51063-71; Volm et al., Eur J. Cancer. [1997] 33:691-3; Kornmann et al., Pancreas. [1998] 17:169-75).

The biological effects of FGFs are mediated by four structurally related tyrosine kinase receptors, denoted FGFR1 to 4, which display broad expression patterns. FGFR1 is the main FGFR expressed on endothelial cells in vitro and has also been detected on activated endothelial cells in vivo (Arbeit et al., Oncogene. [1996] 13:1847-57). Heparan sulphate proteoglycans (HSPGs) serve as co-receptors for FGFs and modulate their effects. FGFs bind with high affinity to HSPGs, which are located on the surface of most cells and within the extracellular matrix.

Five distinct Fibroblast Growth Factor Receptors (FGFRs) have been identified, FGFR1-4 are transmembrane protein kinases while FGFR5 appears to be a soluble receptor. The FGFR extracellular domain consists of three immunoglobulin-like (Ig-like) domains (D1, D2 and D3) containing a heparin binding domain and an acidic box. Alternative splicing of the FGFR mRNAs generates different receptor variants, including the FGFR3IIIb and FGFR3IIIc forms, each having unique 30 ligand specificity.

Heparan sulphate (HS) proteoglycans, which consist of a core protein with covalently attached glycosaminoglycan side chains (GAGs), are found in most mammalian cells and tissues. While the protein part determines the localisation of the proteoglycan in the cell membrane or in the extracellular matrix, the glycosaminoglycan component mediates interactions with a variety of extracellular ligands, such as growth factors, chemokines and adhesion molecules. The biosynthesis of proteoglycans has previously been extensively reviewed. Major groups of the cell surface proteoglycans are the syndecan family of transmembrane proteins (four members in mammals) and the glypican family of proteins attached to the cell membrane by a glycosylphosphatidylinositol (GPI) tail (six members in mammals). The majority of the GAG chains added to the syndecan core proteins through a tetrasaccharide linkage region onto particular serines are HS chains. Although the amino acid sequences of the extracellular domains of specific syndecan types are not conserved among different species, contrary to the transmembrane and the cytoplasmic domains, the number and the positions of the GAG chains are highly conserved. The structure of the GAGs, however, is species-specific and is, moreover, dependent upon the nature of the HSPG-expressing tissue.

Heparan sulphate (HS) is the most abundant member of the glycosaminoglycan (GAG) family of linear polysaccharides which also includes heparin, chondroitin sulphate, dermatan sulphate and keratan sulphate. Naturally occurring HS is characterized by a linear chain of 20-100 disaccharide units composed of N-acetyl-D-glucosamine (GlcNAc) and D-glucuronic acid (GlcA) which can be modified to include N- and O-sulphation (6-O and 3-O sulphation of the glucosamine and 2-O sulphation of the uronic acid) as well as epimerisation of B-D-gluronic acid to a-L-iduronic acid (IdoA).

Clusters of N- and O-sulphated sugar residues, separated by regions of low sulphation, are assumed to be mainly responsible for the numerous protein binding and regulatory properties of HS. In addition to the electrostatic interactions of the HS sulphate groups with basic amino acids, van der Waals and hydrophobic interactions are also thought to be involved in protein binding. Furthermore, the presence of the conformationally flexible iduronate residues seems to favour GAG binding to proteins.

Other factors such as the spacing between the protein binding sites play also a critical role in protein-GAG binding interactions: For example gamma interferon dimerisation induced by HS requires GAG chains with two protein binding sequences separated by a 7 kDa region with low sulphation. Additional sequences are sometimes required for full biological activity of some ligands: in order to support FGF-2 signal transduction, HS must have both the minimum binding sequence as well as additional residues that are supposed to interact with the FGF receptor. Heparin binding proteins often contain consensus sequences consisting of clusters of basic amino acid residues. Lysine, arginine, asparagine, histidine and glutamine are frequently involved in electrostatic contacts with the sulphate and carboxyl groups on the GAG. The spacing of the basic amino acids, sometimes determined by the proteins 3-D structure, are assumed to control the GAG binding specificity and affinity. The biological activity of the ligand can also be affected by the kinetics of HS-protein interaction. Reducing the dimension of growth factor diffusion is one of the suggested HSPG functions for which the long repetitive character of the GAG chains as well as their relatively fast on and off rates of protein binding are ideally suited. In some cases, kinetics rather than thermodynamics drives the physiological function of HS-protein binding. Most HS ligands require GAG sequences of well-defined length and structure. Heparin, which is produced by mast cells, is structurally very similar to heparan sulphate but is characterized by higher levels of post-polymerisation modifications resulting in a uniformly high degree of sulphation with a relatively small degree of structural diversity. Thus, the highly modified blocks in heparan sulphate are sometimes referred to as heparin-like.

Upon binding to a proteoglycan, the affinity of FGF2 to its receptor is enhanced. At present, there exist two structural models of the FGF2-FGFR interaction involving HSPGs. In the first model, the “symmetric model”, Mohammadi et al. describe the formation of a dimerized triple-complex consisting of 2 FGF2, 2 FGFR and 2 HSPG molecules, whereas in the “asymmetric model” Pellegrini et al. propose only one proteoglycan molecule to cross-link the two ligand-receptor complexes forming a heteropentamer (Mohammadi et al., Curr Opin Struct Biol. [2005]15:506-16; Pellegrini et al., Nature [2000] 407:1029-34). In either case, both molecules, FGF2 and FGFR, are involved in HSPGs binding. Our approach to inhibit FGF2-mediated angiogenesis and therefore tumor-growth is, to make dominant negative, soluble FGFR1-mutants with improved affinity for glycosaminoglycans, like displayed on HSPGs, and knocked-out signalling activity that replace wild-type FGFR1 in the signalling complex.

Potzinger et al. (Biochem. Society, 2006, 34, Pt. 3, 435-437) describe the development of chemokine mutants with increased GAG binding affinity and knocked-out GPCR activity for use in anti-inflammatory treatment. SDF-1 variants with increased GAG binding affinity are disclosed in EP2053060.

Wagner et al. (Gastroenterology, 1998, 114, 798-807) disclose suppression of fibroblast growth factor signalling which inhibits cancer growth.

There is a high demand for novel proteins having improved GAG binding characteristic as well as providing antagonistic characteristics specifically in the field of tumor treatment and prevention of metastasis. Therefore it is an object of the invention to provide proteins which can interrupt the closed circuit between FGFR1/FGF2/GAGs and thus prevent target cells from downstream signalling.

### SHORT DESCRIPTION OF THE INVENTION

The object of the invention is achieved by providing the embodiments described in the claims. Accordingly there is provided a soluble extracellular FGFR1 mutant protein with increased GAG binding affinity compared to wild type FGFR1 protein wherein said protein is modified in a structure-conserving way by insertion of at least one basic amino acid or replacement of at least one, specifically of at least two non-basic amino acids by at least one, specifically of at least two basic amino acids.

According to a specific embodiment, the GAG binding region of FGFR1 is modified.

The invention specifically provides a FGFR1 mutant protein wherein at least one, preferably at least two amino acid residues are inserted and/or replaced by lysine, arginine, or histidine.

According to the invention, the mutant FGFR1 protein preferably comprises the D2 domain either alone or in combination with the D3 domain of FGFR1 or at least part thereof.

More specifically, the amino acid replacement and/or insertion is in the C-terminal region of the D2 domain, preferably between amino acids positions 206 and 248 of SEQ ID No. 1, more preferred between amino acid positions 209 and 219.

More specifically, the amino acid replacement may be a heterofunctional amino acid substitution. The inventive FGFR1 mutant protein may consist of up to 354 amino acids, however, additional amino acid sequences like e.g. linker or marker sequences or histidine tags can be linked to said protein thus leading to a protein of more than 354 amino acids.

The insertion or replacement of the amino acid residues can be at any position within the protein, preferably said modification is in a structure conserving way which is defined as a deviation of the modified structure from the soluble part of wild type FGFR1 conformation of less than 30%, preferably less than 20% as measured by far-UV CD spectroscopy.

More specifically, the inventive soluble FGFR1 mutant protein comprises the amino acid sequence

TKPNRMPVAPY WTSPE(X1)ₙME(X2)ₘK LHAVPAA(X3)ₚTV
(X4)_{q} F(X5)ᵣCPSSGTP NPTLRWLKNG KEFKPDHRIG
GYKVR(X6)ₛATWS I(X7)ₜM(X8)ᵤSVVPSDKGNYT
CIVEN EYGSINHTYQ LDVV

wherein
X1, X2, X3, X4 and X5, is of the natural amino acid lysine or of histidine, or arginine, and
X6 is of the natural amino acid tyrosine or any one of histidine, lysine, or arginine, and
X7 is of the natural amino acid isoleucine, or any one of histidine, lysine, or arginine, and
X8 is of the natural amino acid aspartate or any one of histidine, lysine, or arginine, and
wherein any one of m, n, p, q, r, s, t, u is either 1 or 2, and wherein X1-X8 may be the natural amino acid and/or any of the alternative amino acids,
and wherein at least two amino acid residues of the wild type sequence SEQ ID No. 1 are replaced by histidine, lysine or arginine
and optionally wherein up to 2, specifically up to 3, specifically up to 4, specifically more than 4 additional amino acids are altered in a structure-conserving way.

As alternative embodiment, a soluble FGFR1 mutant protein is provided wherein the amino acid sequence of the modified FGFR1 molecule is described by the general formula:

TKPNRMPVAPY WTSPE(X1)ₙME(X2)ₘK LHAVPAA(X3)ₚTV
(X4)_{q} F(X5)ᵣCPSSGTP NPTLRWLKNG KEFKPDHRIG
GYKVR(X6)ₛATWS I(X7)ₜM(X8)ᵤSVVPSD KGNYTCIVEN
EYGSINHTYQ LDVVERSPHR PILQAGLPAN KTVALGSNVE
FMCKVYSDPQ PHIQWLKHIE VNGSKIGPDN LPYVQILKTA
GVNTTDKEME VLHLRNVSFE DAGEYTCLAG NSIGLSHHSA
WLTVLEALEE

wherein
X1, X2, X3, X4 and X5, is of the natural amino acid lysine or any of histidine, or arginine, and
X6 is of the natural amino acid tyrosine or any one of histidine, lysine, or arginine, and
X7 is of the natural amino acid isoleucine, or any one of histidine, lysine, or arginine, and
X8 is of the natural amino acid aspartate or any one of histidine, lysine, or arginine, and
wherein any one of m, n, p, q, r, s, t, u is either 1 or 2, and wherein either of X1-X8 may be the natural amino acid and/or any of the alternative amino acids,
and wherein at least two amino acid residues of the wild type sequence SEQ ID No. 1 are replaced by histidine, lysine or arginine and optionally
wherein up to 2, specifically up to 3, specifically up to 4, specifically more than 4 additional amino acids are altered in a structure-conserving way.

Specifically, at least two amino acid residues of X1 to X8 of the wild type sequence are replaced by histidine, lysine or arginine. More specifically, at least two amino acid residues of X6 to X8 of the wild type sequence are replaced by histidine, lysine or arginine.

The embodiment of the invention specifically covers a FGFR1 mutant protein comprising the sequence of any one of SEQ ID Nos. 4 to 7 or at least part thereof.

Specifically, a FGFR1 mutant protein is provided consisting of any one of sequences SEQ ID Nos. 4 to 7.

More specifically, the FGFR1 protein is free of a His-tag sequence, for example the sequence signal sequence MSHHHHHHSMG (SEQ ID 20) is removed after expression and purification of the inventive protein.

According to a further aspect, the invention refers to an isolated polynucleic acid molecule coding for the inventive FGFR1 protein is provided. More specifically, the isolated polynucleic acid molecule is hybridising to a DNA molecule coding for the inventive protein under stringent conditions.

According to the invention, there is further provided a vector comprising an isolated DNA molecule coding for the inventive FGFR1 protein, and further a recombinant host cell, stably transfected with the vector. Preferably, the recombinant cell is not comprised in a human organism.

The present invention also provides for a pharmaceutical composition, comprising the inventive protein or a polynucleotide coding for the inventive FGFR1 protein or a vector comprising an isolated DNA molecule coding for the inventive FGFR1 and a pharmaceutically acceptable carrier.

The FGFR1 mutant protein or a polynucleotide coding for the inventive FGFR1 protein or a vector comprising an isolated DNA molecule coding for the inventive FGFR1 can also be used in a method for inhibiting or suppressing the biological activity of the respective wild-type protein.

According to a further embodiment, the FGFR1 mutant protein or a polynucleotide coding for the inventive FGFR1 protein or a vector comprising an isolated DNA molecule coding for the inventive FGFR1 can be used for preparing a medicament to be used in the prevention and treatment of angiogenesis, preferably for the treatment of cancer, preferably for the inhibition or decrease or retention of tumour growth and spreading processes.

### FIGURES

FIG. 1: Amino acid sequence of wt FGFR1 protein (SEQ ID 1)

FIG. 2: Sequence of FGFR1 mutants. The first 9 amino acids HHHHHHSMG (SEQ ID 19) represent a linker peptide for affinity purification purposes. Mutations with respect to the wild type chemokine are underlined.

FIG. 3: Affinity increase as measured by SPR/BiaCore (ligand: LMW heparin) and Scatchard Plot analysis.

FIG. 4: pHAT2 expression vector comprising wt FGFR1 nucleotide (SEQ ID No. 8)

FIG. 5: pHAT2 expression vector comprising PA1101 nucleotide (SEQ ID No. 9)

FIG. 6: pHAT2 expression vector comprising PA1102 nucleotide (SEQ ID No. 10)

FIG. 7: pHAT2 expression vector comprising PA1103 nucleotide (SEQ ID No. 11)

FIG. 8: pHAT2 expression vector comprising PA1104 nucleotide (SEQ ID No. 12)

FIG. 9: Inhibition of VEGF or FGF2-induced HUVEC cell sprouting by FGFR1 mutants.

### DETAILED DESCRIPTION OF THE INVENTION

A soluble extracellular FGFR1 mutant protein with increased GAG binding affinity compared to wild type FGFR1 protein is provided wherein the GAG binding region of said protein is modified by insertion of at least one basic amino acid and/or replacement of at least one non-basic amino acid by at least one basic amino acids.

Specifically, said modification is in a structure conserving way.

According to a specific embodiment, the inventive FGFR1 mutant comprises a replacement of at least two non-basic amino acids by at least two basic amino acids.

The term “soluble extracellular” with respect to a portion of the FGFR1 protein or “soluble FGFR1” as used herein shall mean that the FGFR1 protein lacking all of or parts of the transmembrane and intracellular regions, thus, resulting in an FGFR1 which is not attached to cellular membranes.

The soluble FGFR1 protein may be of mammalian origin, for example from feline, canine origin, preferably it is of human origin.

Soluble FGFR1 receptors can be generated by recombinant methods wherein the sequence is expressed by expression systems well known in the art. The receptors may, however, also be generated through natural splicing or proteolytic cleavage which may also lead to ectodomain shedding or by any known genetic or chemical method known by the skilled person to modify certain regions of a protein. Such soluble proteins may also be generated by alternative use of slightly different splice sites at the exon/intron boundaries of the exons encoding the acidic box domain, the membrane-proximal domain or the juxtamembrane domain.

According to a specific embodiment of the invention, the soluble extracellular FGFR1 mutant protein contains the D2 domain (Ig domain II) or the D3 domain (Ig domain III) or the D2/D3 domain or at least parts, fragments or derivatives thereof, provided that the parts, fragments or derivatives still comprise a functional GAG binding region, preferably as determined by a suitable GAG binding assay, for example a Biacore Assay.

The amino acid replacement may be either a heterofunctional or an isofunctional replacement.

The terms “replacement” and “substitution” with respect to amino acids in an amino acid sequence can be used interchangeably according to the invention.

The amino acid replacement may specifically be a heterofunctional amino acid replacement. The term “replacement of a heterofunctional amino acid” is defined as replacement or substitution of the original non-basic amino acid by one or more basic amino acids.

For example, any of amino acids tyrosine, isoleucine or aspartic acid, glycine, alanine, valine, leucine, proline, phenylalanine, methionine, tryptophan, serine, threonine, cysteine, asparagine or glutamine may be replaced by either arginine, lysine or histidine, specifically it it replaced by arginine.

Preferably, tyrosine, isoleucine or aspartic acid may be replaced by arginine, lysine or histidine, specifically it is replaced by arginine.

It was surprisingly shown that specifically one, two, three, four, five or more heterofunctional amino acid replacements result in even more increased GAG binding affinity of the so modified FGFR1 protein compared to isofunctional amino acid substitution wherein a basic amino acid is substituted by another basic amino acid with the same or increased basic character, i.e. substituting lys residues by arg residues.

It has been shown that increased GAG binding affinity can be obtained by increasing the relative amount of basic amino acids in the GAG binding region of a protein (WO 05/054285), leading to a modified protein that acts as competitor or inhibitor of naturally GAG binding proteins. This was particularly shown for interleukin-8. However the specific location of GAG binding regions and the specific disclosure of selectively introducing basic amino acids was not disclosed for the FGFR1 protein.

The present invention is specifically based on engineering a higher GAG binding affinity into the soluble extracellular form of human FGFR1. This is accomplished by introducing basic amino acids into the GAG binding region of the protein, specifically by inserting basic amino acids or by replacing non-basic amino acids by basic amino acids in the D2 or D2/D3 domains of FGFR1.

Said FGFR1 mutants preferably exhibit a conserved amino acid sequence, the secondary and/or tertiary protein structure shall be conserved with respect to the sequence or domains of the soluble part of unmodified FGFR1 sequence which are not involved in GAG binding, specifically with respect to the D2 and D3 domains, but have an improved affinity for glycosaminoglycans, for example heparin, heparan sulfate, chondroitin sulfate, keratin sulfate or dermatan sulfate compared to wtFGFR1.

A further subject matter of the present invention is to inhibit FGF2-mediated signalling by antagonising the GAG interaction in the FGF2-FGFR1-GAG complex with the modified soluble form of FGFR1 which may be specifically useful in the context of therapeutic approaches especially for the prevention and treatment of tumor growth and spreading processes.

Such recombinant mutant proteins are expected to act as inhibitor or antagonists disrupting the natural FGF2/FGFR1/GAG triple complex by competing with the wtFGFR1 for GAG binding and thus pulling out the GAG ligand required for signalling. By this means, FGF2 signalling is interrupted since the mutant FGFR1 protein lacks the intracellular signalling domain in addition to displaying increased GAG binding affinity.

The present invention specifically provides soluble FGFR1 variant proteins preferably from human origin consisting of the extracellular domain or part thereof with increased GAG binding affinity which are signalling deficient compared to wtFGFR1 due to their lack or inhibition or down-regulation of functional tyrosine kinase domain.

Rational design of FGFR1 mutants with respect to increased GAG binding affinity is done in a structure-conserving way either (1) by insertion of at least one basic amino acid and/or (2) by replacement of at least one non-basic amino acid by at least one basic amino acid or (3) by replacing more than one, preferably two, three, four, five or more residues by arg, lys or his residues to result in a higher pKa value of the latter amino acids or a combination of said methods.

Preferably, at least two, more preferably at least three, more preferably at least four amino acids are replaced by basic amino acids within the D2 or D2/D3 domains. Alternatively, 5, 6, 7 or even more amino acids but preferably not more than 10 amino acids can be inserted or replaced within the D2 or D2/D3 domains by basic amino acids.

Alternatively, at least two, specifically at least three, specifically at least four, five, six, seven, eight or nine basic amino acids can be inserted in the protein to increase GAG binding affinity compared to the wt FGFR1 protein. According to an embodiment, not more than ten amino acids, more preferred not more than eight amino acids, even more preferred not more than six amino acids can be inserted in the protein to increase GAG binding affinity compared to the wt FGFR1 protein.

According to the invention, the soluble FGFR1 mutant protein consists of up to approx. 354 amino acids covering the extracellular domain of the FGFR protein. According to the definition of the present invention also variants or fragments of reduced length of amino acids are covered if these fragments are functionally equivalent, i.e. they still comprise a GAG binding region and show inhibited FGF2-mediated signalling by antagonising the GAG interaction.

According to the terms of the invention, “fragments” or “derivatives” or “parts” of the FGFR1 mutant proteins are also covered by the invention given that their GAG binding capabilities are not adversely affected compared to the FGFR1 mutant protein comprising the insertion or replacements as covered by the invention but lacking any further modifications.

In a specific embodiment, the functionally active derivative or fragment according to the invention due to amino acid exchanges, deletions or insertions may conserve, or more preferably improve, the structural stability.

More specifically the soluble FGFR1 mutant protein comprises at least amino acids from amino acid position 22 to 376 according to the numbering of SEQ ID No. 1.

Even more preferred the soluble FGFR1 mutant protein consists of the D2 domain or the D2/D3 domains or part thereof which is modified according to the invention.

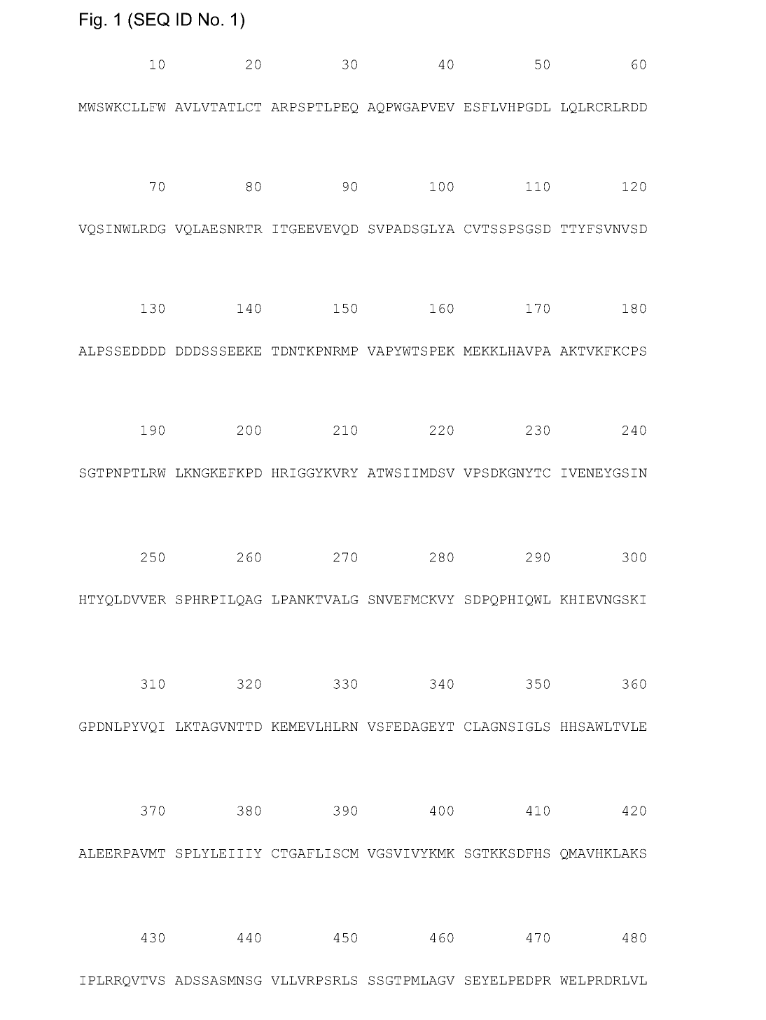

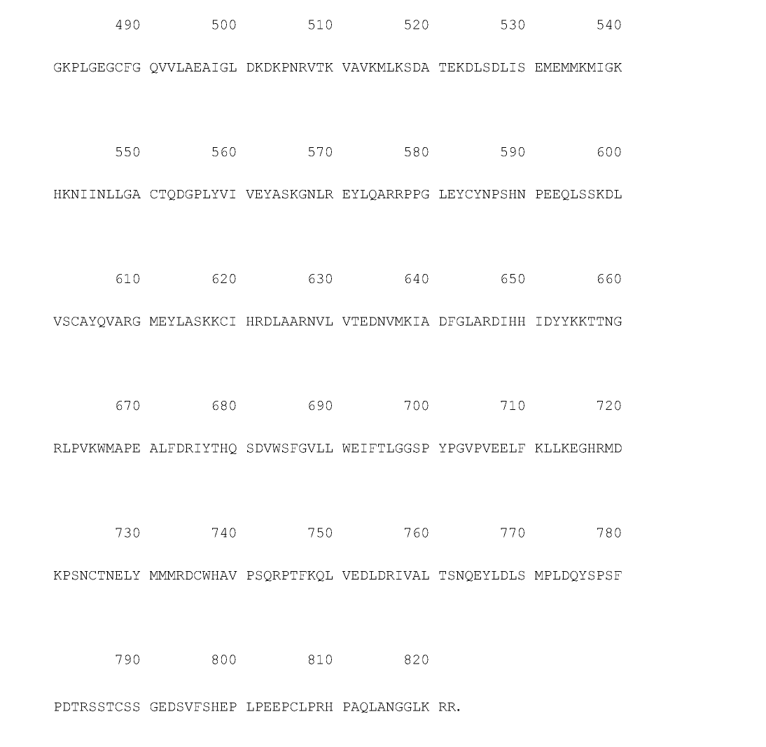

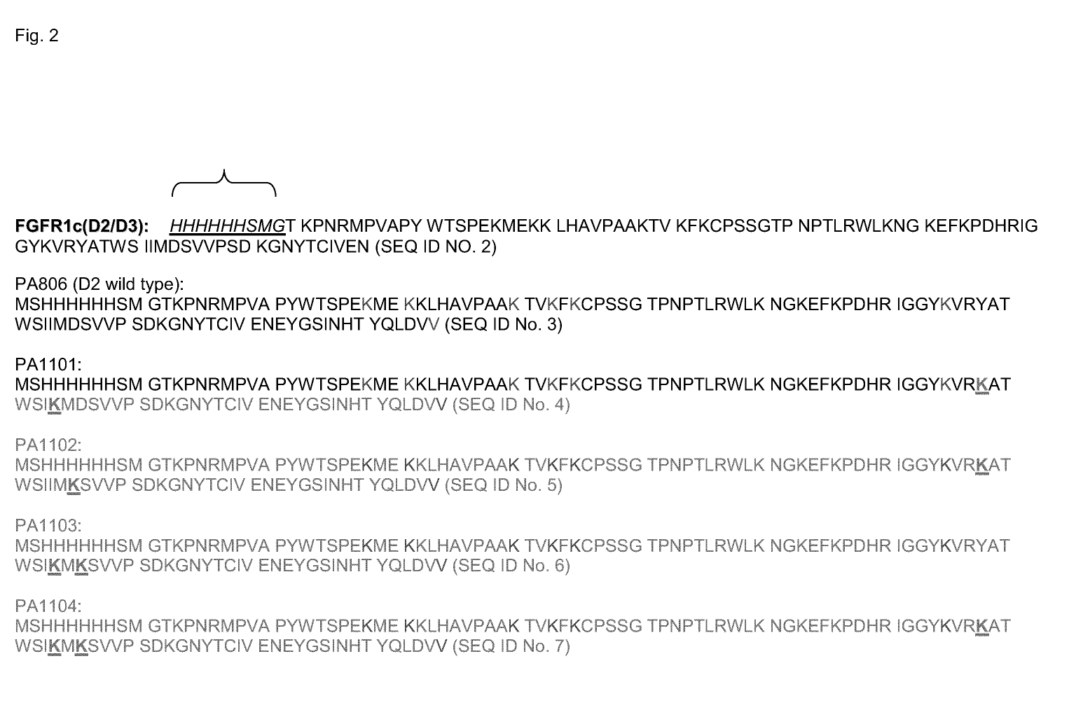

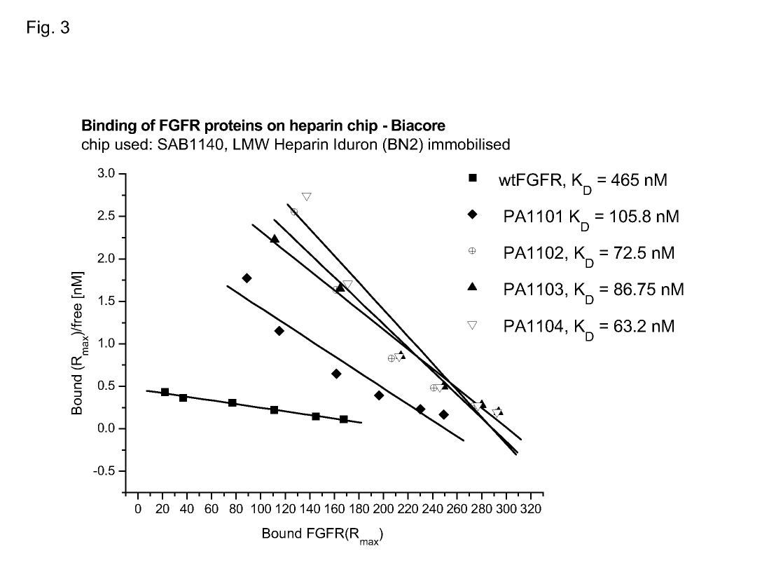

The wild type FGFR1D2/D3 domain is defined as consisting of amino acids from position 144 to 364 of the wild type FGF1 receptor sequence according to the amino acid numbering of SEQ ID No. 1 (FIG. 1).

The wild type FGFR1D2 domain is defined as consisting of the amino acids from position 144 to 265 of the wild type FGF1 receptor sequence according to the amino acid numbering of SEQ ID No. 1 (FIG. 1).

The wild-type FGFR1D3 domain is defined as consisting of the amino acids from position 266 to 364 of the wild type FGF1 receptor sequence according to the amino acid numbering of SEQ ID No. 1 (FIG. 1).

According to a specific embodiment, the amino acid replacement and/or insertion is in the C-terminal region of the D2 domain, preferably between amino acid positions 206 and 248 of SEQ ID No. 1, more preferred between amino acid positions 208 and 227, more preferred between 209 and 219. Even more specific, single or combined amino acid deletions, substitutions or insertions can be at amino acid positions 210, 216 and/or 218 according to the numbering of SEQ ID. No. 1. In addition, further amino acid positions, for example, but not limited to, positions 163, 164, 175, 207, 211, or 265 can be modified by replacing or otherwise introducing the amino acids by arg, lys, or his.

According to an alternative embodiment of the invention, the soluble FGFR1 mutant can be a polypeptide sequence comprising or consisting of the D2 domain consisting of the amino acids 144 to 260 of SEQ ID No. 1 wherein said D2 domain is modified according to the invention to increase GAG binding affinity of said polypeptide.

More specifically, the soluble FGFR1 mutant comprises amino acid replacements at positions 210 and 218, or at positions 216 and 218 or alternatively at all positions 210, 216 and 218. More specifically the amino acids at these positions are replaced by arg. A protocol for introducing or improving a GAG binding region is for example described in WO 05/054285 and can be as follows:

Identifying a region of the protein which is involved in GAG binding

Designing a new GAG binding site by introducing (replacement or insertion) basic amino acids, arg, lys, and his, residues at any position or by deleting amino acids which interfere with GAG binding

Checking the conformational stability of the resulting mutant protein in silico

Cloning the wild-type protein cDNA (alternatively: purchase the cDNA)

Using this as template for PCR-assisted mutagenesis to introduce the above mentioned changes into the amino acid sequence

Subcloning the mutant gene into a suitable expression system (prokaryotic or eukaryotic dependent upon biologically required post-translational modifications)

Expressing, purifying and characterizing of the mutant protein in vitro Criterion for an increased GAG binding affinity: K_{d}^{GAG}(mutant)≦10 μM.

Checking for structural conservation by far-UV CD spectroscopy or 1-D NMR spectroscopy.

A deviation of the modified structure as measured by far-UV CD spectroscopy from wild type structure of less than 30%, preferably less than 20% is defined as structure conserving modification according to the invention. According to the present invention the FGFR1 mutant protein can comprise a modification in a structure conserving way wherein the deviation of the modified structure as measured by far-UV CD spectroscopy from wild type FGFR1 structure is less than 30%, preferably less than 20%.

As used herein, the term “GAG binding region” means the native GAG binding site including N- and C-terminal regions adjacent to the GAG binding site and regions within the structural vicinity of a native GAG binding site.

The term “vicinity” as defined according to the invention comprises amino acid residues which are located within the conformational neighbourhood of the GAG binding site, but not positioned at the GAG binding sites. Conformational neighbourhood can be defined either as amino acid residues which are located adjacent, e.g. within 5, 6, 7, 8, 9 or 10 amino acids adjacent to GAG binding amino acid residues in the amino acid sequence of a protein, or amino acids which are conformationally adjacent due to three dimensional structure or folding of the protein.

The term “adjacent” as used herein shall mean as lying within the cut-off radius of the respective amino acid residues to be modified of not more than 20 nm, preferably 15 nm, preferably 10 nm, preferably 5 nm.

To be able to perform their biological function, proteins fold into one, or more, specific spatial conformations, driven by a number of non-covalent interactions such as hydrogen bonding, ionic interactions, Van der Waals' forces and hydrophobic packing. Three dimensional structure can be determined by known methods like X-ray crystallography or NMR spectroscopy.

Identification of native GAG binding sites can be determined by mutagenesis experiments. GAG binding sites of proteins are characterized by basic residues located at the surface of the proteins. To test whether these regions define a GAG binding site, these basic amino acid residues can be mutagenized and the decrease of heparin binding affinity can be measured. This can be performed by any affinity measurement techniques as known in the art, e.g. isothermal fluorescence titration, surface plasmon resonance, isothermal titration calorimetry test.

Rational designed mutagenesis by insertion or substitution of basic amino acids can be performed to introduce foreign amino acids in the vicinity of the native GAG binding sites which can result in an increased size of the GAG binding site and in an increase of GAG binding affinity.

The GAG binding site or the vicinity of said site can also be determined by using a method as described in detail in WO2010122176 comprising:

(a) providing a complex comprising the protein and the GAG ligand molecule, for example heparan sulfate (HS), heparin, keratin sulfate, chondroitin sulfate, dermatan sulfate and hyaluronic acid etc. bound to said protein;
(b) contacting said complex with a cleavage reagent like a protease, e.g. trypsin, capable of cleaving the protein, wherein said GAG ligand molecule blocks protein cleavage in a region of the protein where the GAG ligand molecule is bound, and whereby said protein is cleaved in regions that are not blocked by said bound GAG ligand molecule; and
(c) separating and detecting the cleaved peptides, wherein the absence of cleavage events in a region of the protein indicates that said GAG ligand molecule is bound to that region. Detection can be for example by LC-MS, nano HPLC-MS/MS or Mass Spectrometric Methods.

The basic amino acids according to the invention are of the group consisting of arg, lys, his.

According to the invention the soluble FGFR1 mutant protein can additionally comprise further amino acid residues on the C- or N-terminus of the protein, which do not result in or otherwise interfere with the signalling activity of the protein.

These additional amino acids can be useful as protection sequences or amino acids or peptides are attached to the D2 or D2D3 domains which can serve as linker or marker or stabilizing peptides for detection or purification or stabilization. For example, an affinity tag linker region like for example a His tag linker can be attached. The linker sequences can be of up to 50 amino acids length, preferably of up to 30 amino acids, preferably up to 10 amino acids. Alternatively, the protein can be PEGylated to increase its half-life by covalent attachment of poly(ethylene glycol) polymer chains to the molecule. Alternatively they may be coupled to carrier proteins.

Even more specifically, the FGFR1 mutant consists of 116 amino acids or, in an alternative embodiment, of 132 amino acids due to the presence of a linker linking the D2 and D3 domains which may increase stability of said mutant.

According to a specific embodiment of the invention the soluble FGFR1 mutant protein is stable of at least about 3 months, preferably for at least about 6 months at −20° C.

According to the present invention, the binding affinity to GAG proteins (e.g. heparin or heparin sulfate) of the mutant FGFR1 protein of the invention as represented by a specific Kd value is at least 1.5-fold improved, preferably at least 2.5-fold, more preferred at least 5 fold compared to soluble wtFGFR1 protein, specifically measured in a saturation assay

Alternatively, the amino acid sequence of the inventive FGFR1 mutant protein can be described by the general formula

TKPNRMPVAPY WTSPE(X1)ₙME(X2)ₘK LHAVPAA(X3)ₚTV
(X4)_{q} F(X5)ᵣCPSSGTP NPTLRWLKNG KEFKPDHRIG
GYKVR(X6)ₛATWS I(X7)ₜM(X8)ᵤSVVPSD KGNYTCIVEN
EYGSINHTYQ LDVV

wherein
X1, X2, X3, X4 and X5, is of the natural amino acid lysine or any of histidine, or arginine, and
X6 is of the natural amino acid tyrosine or any one of histidine, lysine, or arginine, and
X7 is of the natural amino acid isoleucine, or any one of histidine, lysine, or arginine, and
X8 is of the natural amino acid aspartate or any one of histidine, lysine, or arginine, and
wherein any one of m, n, p, q, r, s, t, u is either 1 or 2, with the proviso that if m, n, p, q, r, s, t, u is 2, either of X1-X8 may be the natural amino acid and/or any of the alternative amino acids,
   and wherein at least two amino acid residues of the wild type sequence SEQ ID No. 1 are replaced by histidine, lysine or arginine (SEQ ID No. 13)
   and optionally wherein up to 2, specifically up to 3, specifically up to 4, specifically more than 4 additional amino acids are altered in a structure-conserving way.

Alternatively, the amino acid sequence of the inventive FGFR1 mutant protein can be described by the general formula

TKPNRMPVAPY WTSPE(X1)ₙME(X2)ₘK LHAVPAA(X3)ₚTV(X4)_{q}
F(X5)ᵣCPSSGTP NPTLRWLKNG KEFKPDHRIG GYKVR
(X6)ₛATVVS I(X7)ₜM(X8)ᵤSVVPSD KGNYTCIVEN
EYGSINHTYQ LDVVERSPHR PILQAGLPAN KTVALGSNVE
FMCKVYSDPQ PHIQWLKHIE VNGSKIGPDN LPYVQILKTA
GVNTTDKEME VLHLRNVSFE DAGEYTCLAG NSIGLSHHSA
WLTVLEALEE

wherein
X1, X2, X3, X4 and X5, is of the natural amino acid lysine or any of histidine, or arginine, and
X6 is of the natural amino acid tyrosine or any one of histidine, lysine, or arginine, and
X7 is of the natural amino acid isoleucine, or any one of histidine, lysine, or arginine, and
X8 is of the natural amino acid aspartate or any one of histidine, lysine, or arginine, and
wherein any one of m, n, p, q, r, s, t, u is either 1 or 2, with the proviso that if m, n, p, q, r, s, t, u is 2, either of X1-X8 may be the natural amino acid and/or any of the alternative amino acids,
and wherein at least two amino acid residues of the wild type sequence SEQ ID No. 1 are replaced by histidine, lysine or arginine. (SEQ ID No. 14) and optionally wherein up to 2, specifically up to 3, specifically up to 4, specifically more than 4 additional amino acids are altered in a structure-conserving way.

A further aspect of the present invention is an isolated polynucleic acid molecule which codes for the inventive protein as described above. The polynucleic acid may be DNA or RNA. Thereby the modifications which lead to the inventive FGFR1 mutant protein are carried out on DNA or RNA level by recombination techniques. This inventive isolated polynucleic acid molecule is suitable for diagnostic methods as well as gene therapy and the production of the inventive FGFR1 mutant protein on a large scale.

Specifically said polynucleic acid molecules are DNA molecules which comprise the following sequences:

((PA1101, SEQ ID No. 15)
ATGAGTCATCACCATCACCATCACTCCATGGGGACCAAACCAAACCGTAT
GCCCGTAGCTCCATATTGGACATCCCCAGAAAAGATGGAAAAGAAATTGC
ATGCAGTGCCGGCTGCCAAGACAGTGAAGTTCAAATGCCCTTCCAGTGGG
ACCCCAAACCCCACACTGCGCTGGTTGAAAAATGGCAAAGAATTCAAACC
TGACCACAGAATTGGAGGCTACAAGGTCCGTAAAGCCACCTGGAGCATCA
AAATGGACTCTGTGGTGCCCTCTGACAAGGGCAACTACACCTGCATTGTG
GAGAATGAGTACGGCAGCATCAACCACACATACCAGCTGGATGTCGTGTA
A
(PA1102, SEQ ID No. 16)
ATGAGTCATCACCATCACCATCACTCCATGGGGACCAAACCAAACCGTAT
GCCCGTAGCTCCATATTGGACATCCCCAGAAAAGATGGAAAAGAAATTGC
ATGCAGTGCCGGCTGCCAAGACAGTGAAGTTCAAATGCCCTTCCAGTGGG
ACCCCAAACCCCACACTGCGCTGGTTGAAAAATGGCAAAGAATTCAAACC
TGACCACAGAATTGGAGGCTACAAGGTCCGTAAAGCCACCTGGAGCATCA
TAATGAAATCTGTGGTGCCCTCTGACAAGGGCAACTACACCTGCATTGTG
GAGAATGAGTACGGCAGCATCAACCACACATACCAGCTGGATGTCGTGTA
A
(PA1103, SEQ ID No. 17)
ATGAGTCATCACCATCACCATCACTCCATGGGGACCAAACCAAACCGTAT
GCCCGTAGCTCCATATTGGACATCCCCAGAAAAGATGGAAAAGAAATTGC
ATGCAGTGCCGGCTGCCAAGACAGTGAAGTTCAAATGCCCTTCCAGTGGG
ACCCCAAACCCCACACTGCGCTGGTTGAAAAATGGCAAAGAATTCAAACC
TGACCACAGAATTGGAGGCTACAAGGTCCGTTATGCCACCTGGAGCATCA
AAATGAAATCTGTGGTGCCCTCTGACAAGGGCAACTACACCTGCATTGTG
GAGAATGAGTACGGCAGCATCAACCACACATACCAGCTGGATGTCGTGTA
A
(PA1104, SEQ ID No 18)
ATGAGTCATCACCATCACCATCACTCCATGGGGACCAAACCAAACCGTAT
GCCCGTAGCTCCATATTGGACATCCCCAGAAAAGATGGAAAAGAAATTGC
ATGCAGTGCCGGCTGCCAAGACAGTGAAGTTCAAATGCCCTTCCAGTGGG
ACCCCAAACCCCACACTGCGCTGGTTGAAAAATGGCAAAGAATTCAAACC
TGACCACAGAATTGGAGGCTACAAGGTCCGTAAAGCCACCTGGAGCATCA
AAATGAAATCTGTGGTGCCCTCTGACAAGGGCAACTACACCTGCATTGTG
GAGAATGAGTACGGCAGCATCAACCACACATACCAGCTGGATGTCGTGTA
A

According to an alternative embodiment, sequences that are at least 95% identical, preferably at least 98%, more preferred at least 99%, more preferred at least 99.5% identical to said sequences are also covered by the invention, which sequences still have the same or improved functional activities, e.g. GAG binding of the encoded proteins.

Still preferred, the isolated polynucleic acid molecule hybridises to the above defined inventive polynucleic acid molecule under stringent conditions. Depending on the hybridisation conditions complementary duplexes form between the two DNA or RNA molecules, either by perfectly matching or also comprising mismatched bases (see Sambrook et al., Molecular Cloning: A laboratory manual, 2^{nd} ed., Cold Spring Harbor, N.Y. 1989). Probes greater in length than about 50 nucleotides may accomplish up to 25 to 30% mismatched bases. Smaller probes will accomplish fewer mismatches. The tendency of a target and probe to form duplexes containing mismatched base pairs is controlled by the stringency of the hybridisation conditions which itself is a function of factors, such as the concentrations of salt or formamide in the hybridisation buffer, the temperature of the hybridisation and the post-hybridisation wash conditions. By applying well known principles that occur in the formation of hybrid duplexes conditions having the desired stringency can be achieved by one skilled in the art by selecting from among a variety of hybridisation buffers, temperatures and wash conditions. Thus, conditions can be selected that permit the detection of either perfectly matching or partially matching hybrid duplexes. The melting temperature (Tm) of a duplex is useful for selecting appropriate hybridisation conditions. Stringent hybridisation conditions for polynucleotide molecules over 200 nucleotides in length typically involve hybridising at a temperature 15-25° C. below the melting temperature of the expected duplex. For oligonucleotide probes over 30 nucleotides which form less stable duplexes than longer probes, stringent hybridisation usually is achieved by hybridising at 5 to 10° C. below the Tm. The Tm of a nucleic acid duplex can be calculated using a formula based on the percent G+C contained in the nucleic acids and that takes chain lengths into account, such as the formula

Tm=81.5−16.6(log [NA⁺])+0.41(% G+C)−(600/N), where N=chain length.

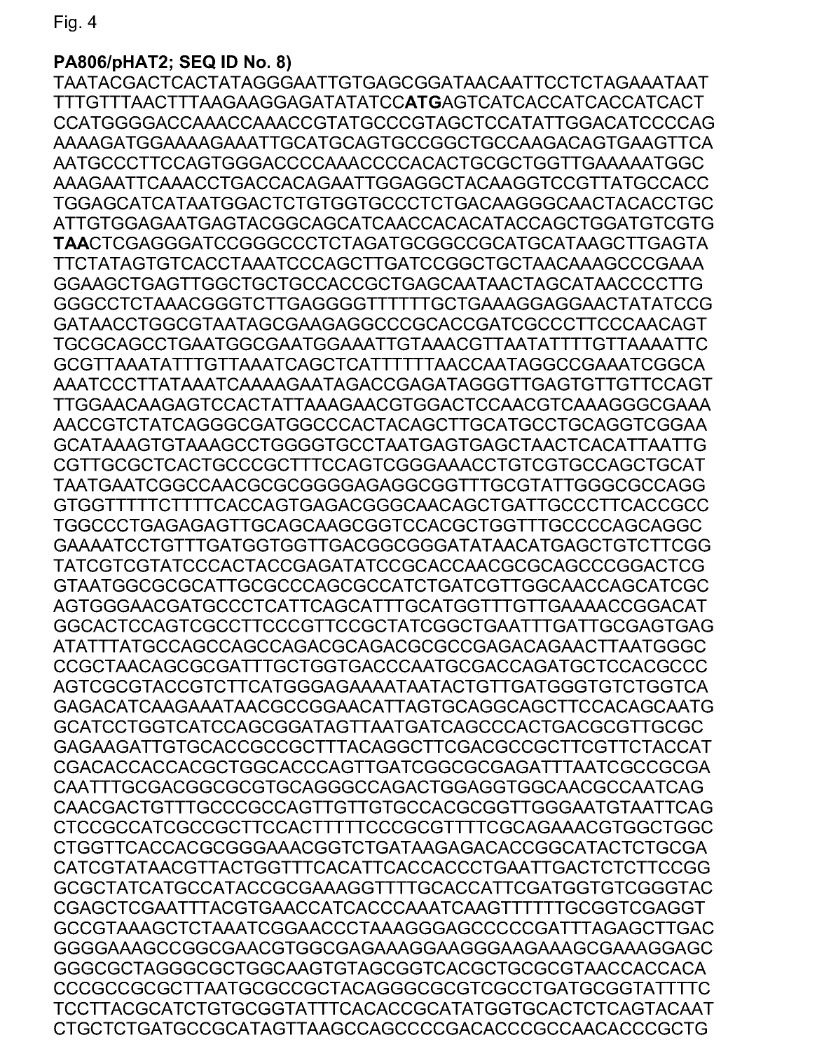

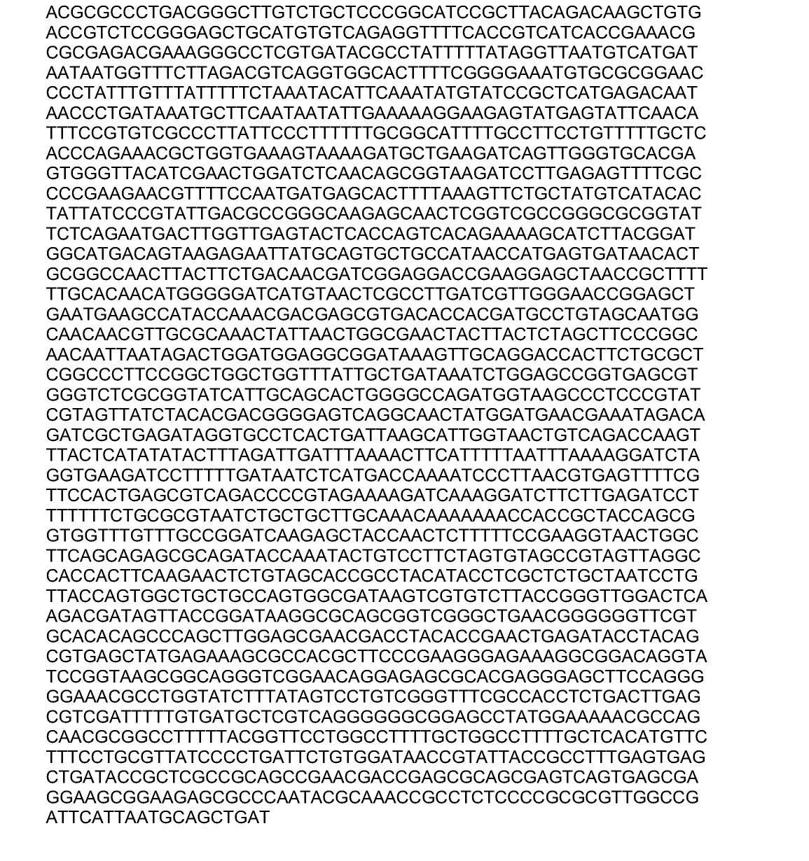

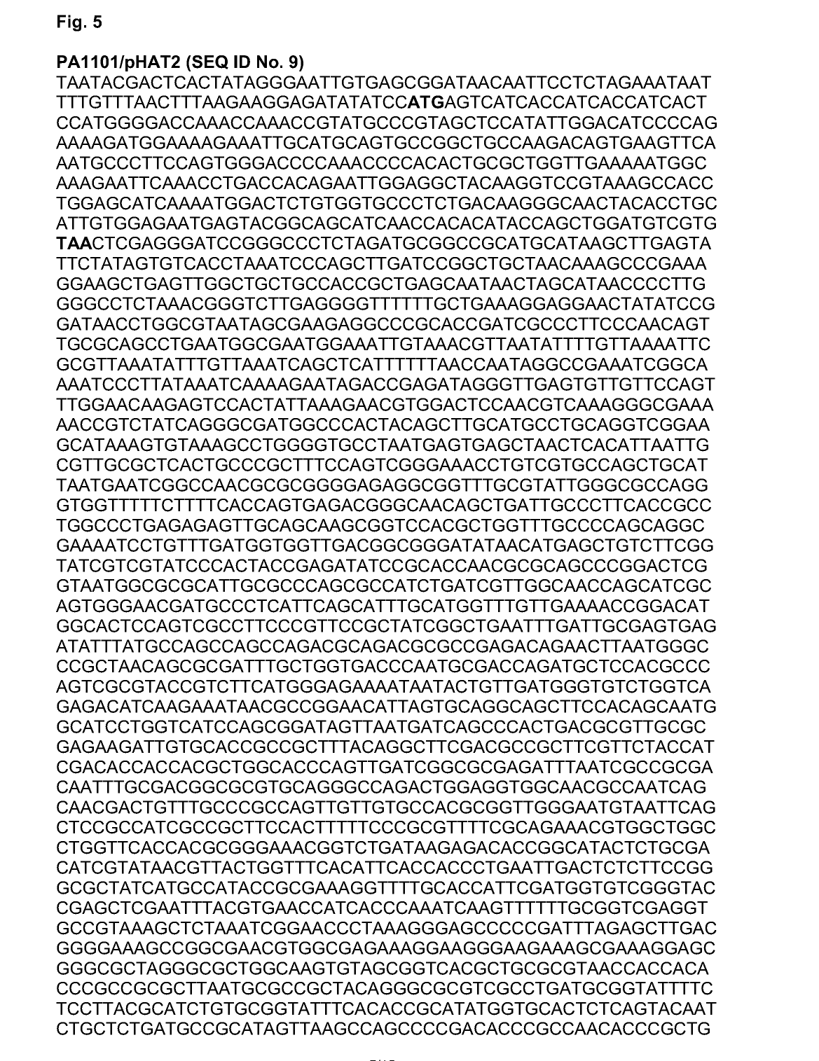

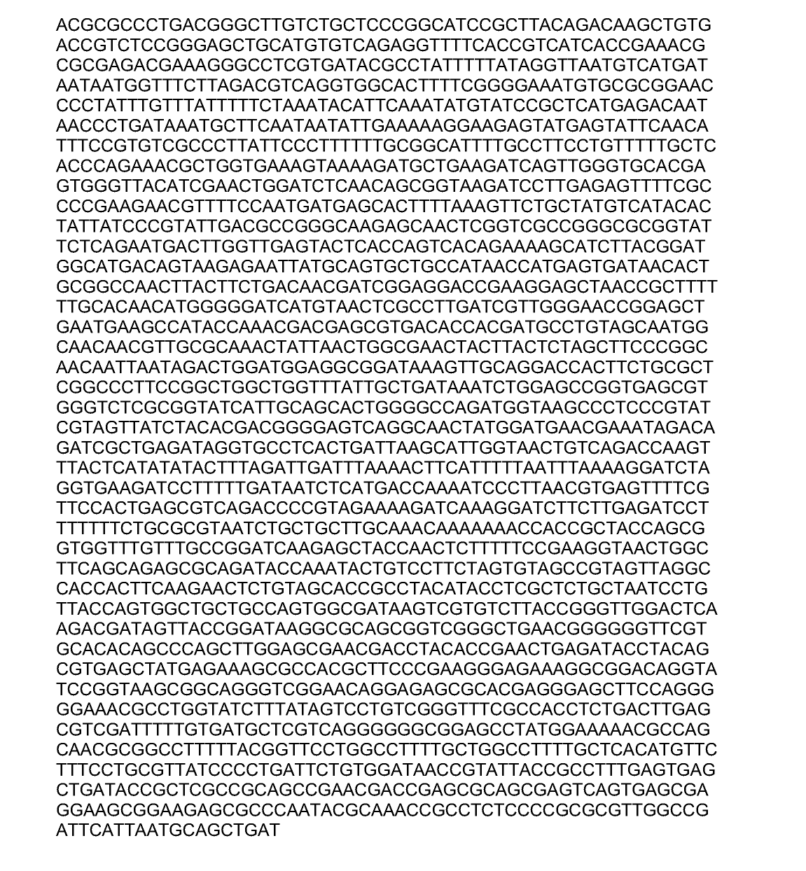

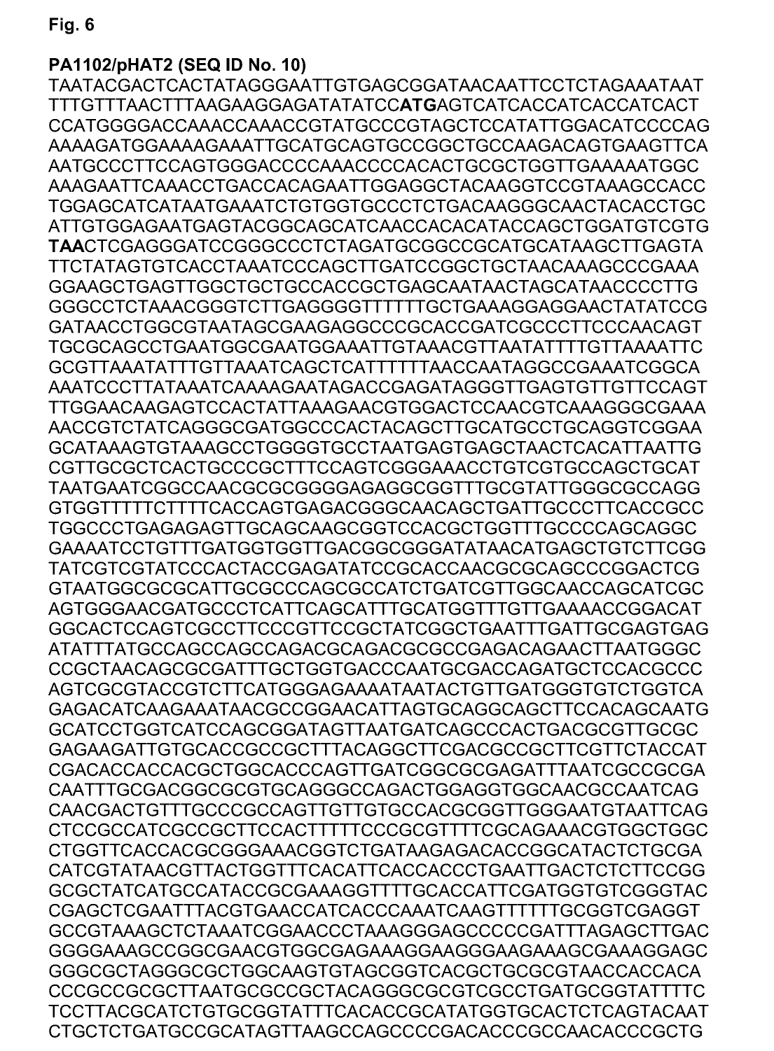

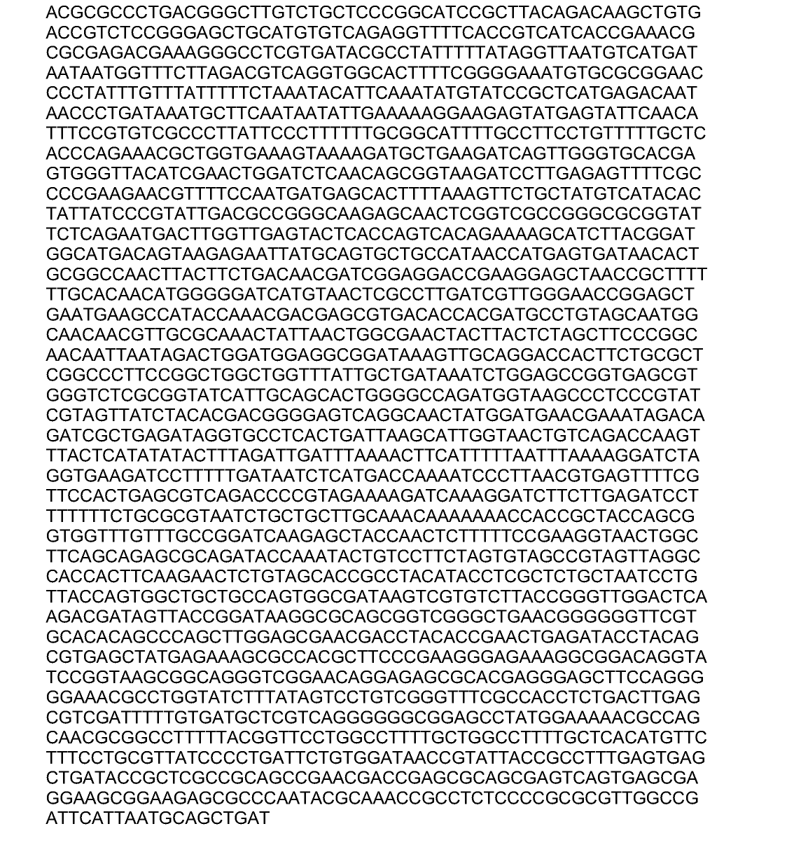

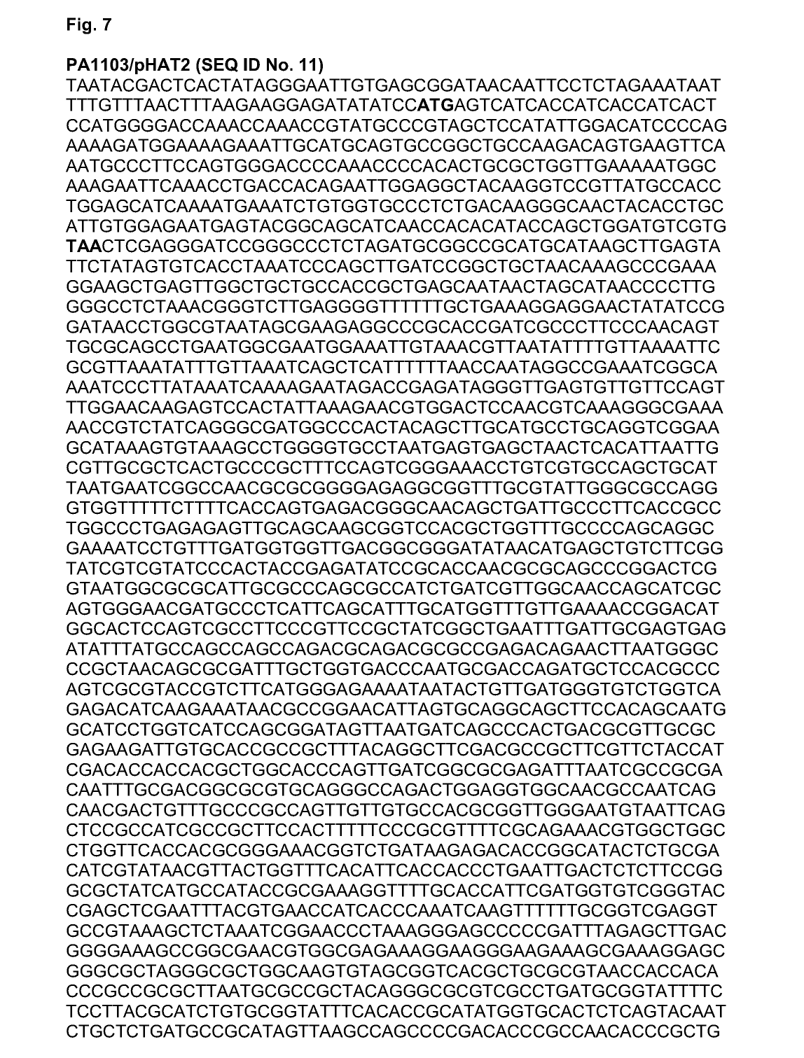

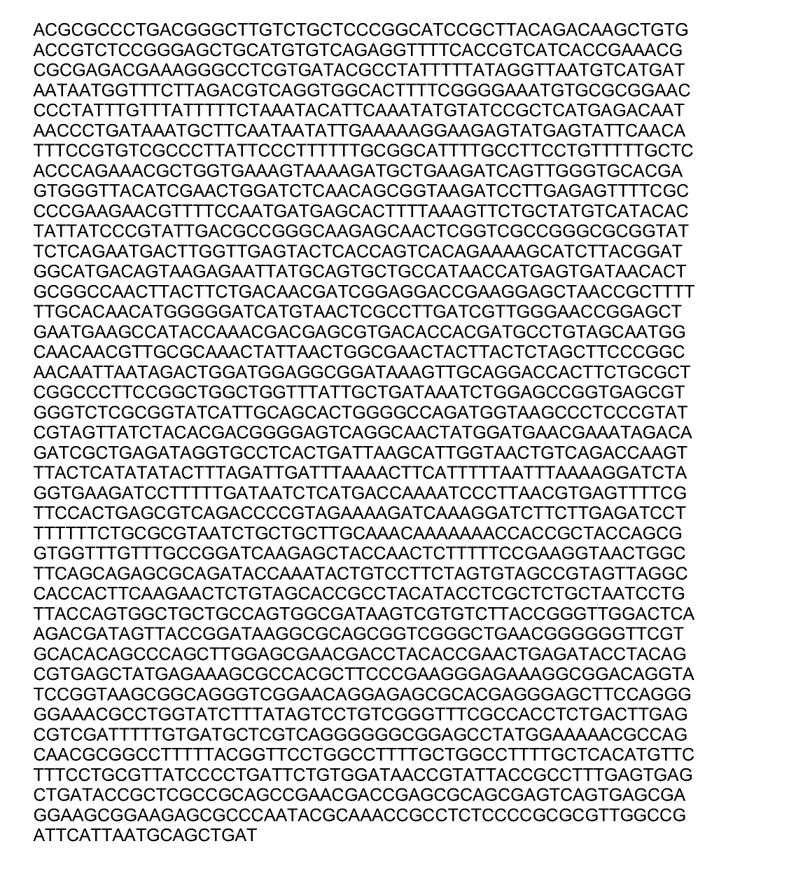

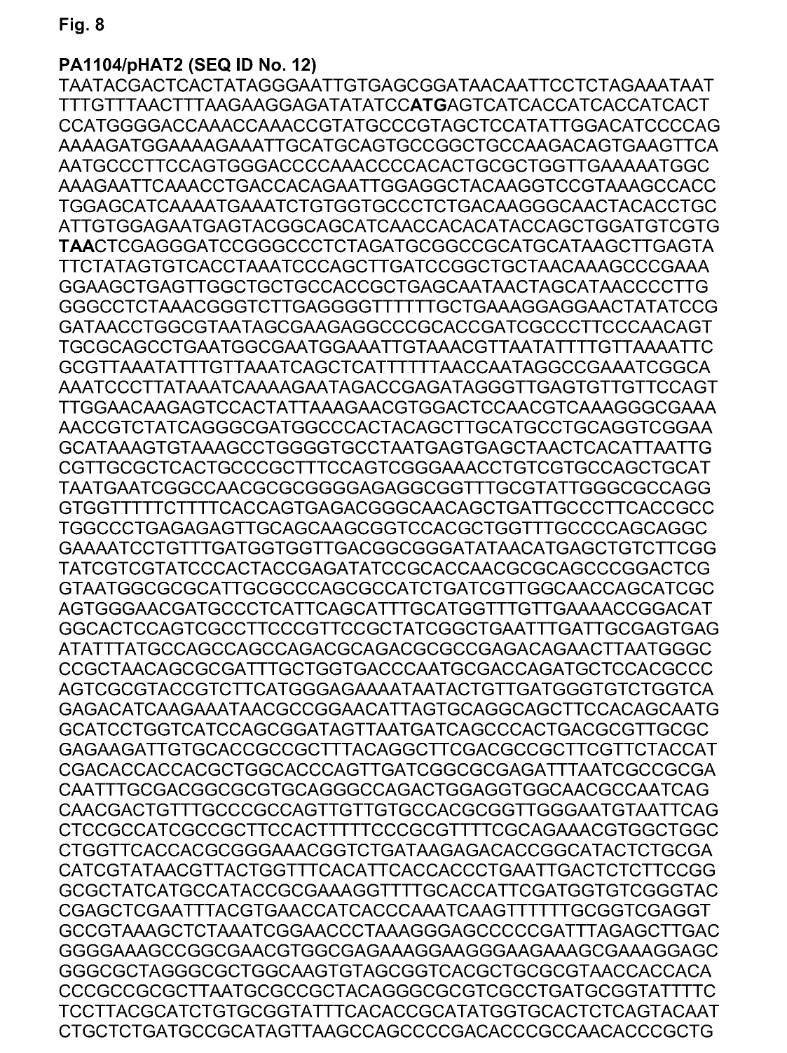

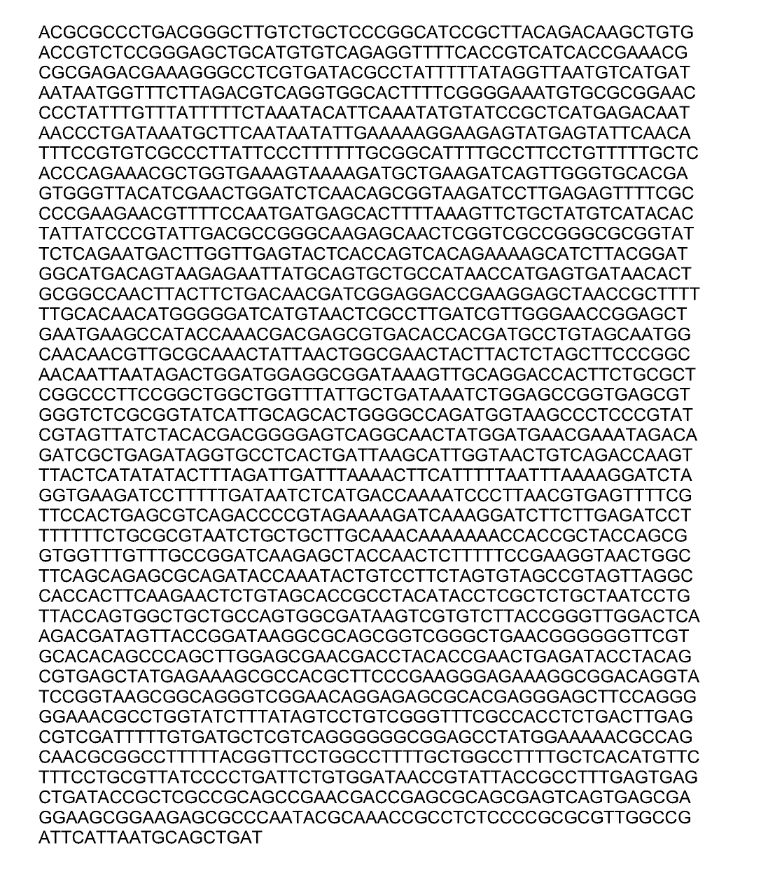

A further aspect relates to a vector comprising an isolated DNA molecule according to the present invention as defined above. The vector comprises all regulatory elements necessary for efficient transfection as well as efficient expression of proteins. Such vectors are well known in the art and any suitable vector can be selected for this purpose. Such vectors may be specifically designed for expression in mammalian cells, for example, but not limited to Vero cells, CHO cells etc. or bacterial cells, for example, but not limited to E. coli cells. Specifically, said vectors are His-tagged expression vectors like pHAT2. The sequence of said vectors expressing FGFR1 mutants are shown in FIGS. 4 to 8.

A further aspect of the present invention relates to a recombinant cell which is transfected with an inventive vector as described above. Transfection of cells and cultivation of recombinant cells can be performed as well known in the art. The recombinant cell is not comprised within the human organism. Such a recombinant cell as well as any therefrom descendant cell comprises the vector. Thereby a cell line is provided which expresses the modified FGFR1 mutant protein either continuously or upon activation depending on the vector.

A further aspect of the invention relates to a pharmaceutical composition comprising a FGFR1 mutant protein, a polynucleic acid or a vector according to the present invention as defined above and a pharmaceutically acceptable carrier. Of course, the pharmaceutical composition may further comprise additional substances which are usually present in pharmaceutical compositions, such as salts, buffers, emulgators, colouring agents, etc.

A further aspect of the present invention relates to the use of the FGFR1 protein, a polynucleic acid or a vector according to the present invention as defined above in a method for inhibiting or suppressing the biological activity of the growth factor ligand, namely FGF2. As mentioned above, the FGFR1 mutant protein of the invention will act as an antagonist of the FGF2 biological activity whereby the side effects which might occur with known proteins are avoided or at least reduced with the inventive FGFR1 mutant protein. In this case this will particularly effect the biological activity involved in cancer development, metastasis and angiogenesis.

Therefore, a further use of the FGFR1 mutant protein, a polynucleic acid or a vector according to the present invention as defined above is in a method for producing a medicament, specifically applicable for the treatment of cancer. In particular, it acts as antagonist without side effects and is particularly suitable for the treatment of patients with cancer disease, specifically for the treatment of angiogenesis during tumor growth or metastatic cancer.

In general all diseases wherein FGF2 and/or FGFR1 are involved may be treated using the inventive soluble FGFR1 mutant protein. Specifically, these diseases may be, but are not limited to artherosclerosis, breast cancer, lung carcinomas: non-small cell lung carcinoma and small cell lung carcinoma bladder cancer, urothelial cell carcinoma—(UCC), prostate cancer-myeloid and lymphoid neoplasms, myeloproliferative syndrome (EMS), rhabdomyosarcoma (RMS), atherosclerosis, brain cancer (astrocytic tumours), esophageal carcinoma, chronic lymphocytic leukemia, hairy cell leukemia, multiple myeloma (MM), non-Hodgkins lymphoma, renal cell carcinoma, pancreatic carcinoma, colorectal cancer, uveal melanoma, involvement in genetic disorders (Kallman syndrome or LADD syndrome, craniosynostosis syndrome), depression, bipolar disorder, schizophrenia, Alzheimer's disease and Huntington's disease.

A further aspect of the present invention is also a method for the treatment of cancer diseases, wherein the FGFR1 mutant protein according to the invention, the isolated polynucleic acid molecule or vector according to the present invention or a pharmaceutical preparation according to the invention is administered to a patient. Due to the antagonistic activity of the mutant FGFR1, the FGF2 mediated angiogenesis can be inhibited. This can be used for the prevention or treatment of cancer diseases, specifically for the prevention and treatment of tumor cell growth and spreading of tumors.

The administration of a composition comprising the FGFR1 mutant proteins of the invention may be by intravenous, intramuscular or subcutaneous route. Other routes of administration, which may establish the desired blood levels of the respective ingredients such as systemic administration or inhalation, are also comprised.

The medicament comprising the composition according to the invention can be formulated together with a pharmaceutically acceptable carrier. Pharmaceutically acceptable is meant to encompass any carrier, which does not interfere with the effectiveness of the biological activity of the active ingredient and that is not toxic to the host to which is administered. For example, for parenteral administration, the modified FGFR1 may be formulated in unit dosage form for injection in vehicles such as saline, dextrose solution, serum albumin and Ringer's solution. Besides the pharmaceutically acceptable carrier also minor amounts of additives, such as stabilisers, excipients, buffers and preservatives can be included.

Besides the pharmaceutically acceptable carrier also minor amounts of additives, such as stabilizers, excipients, buffers and preservatives can be included.

The specific dose of soluble FGFR2 mutant protein, optionally formulated in a suitable manner for administration, administered according to this invention to obtain therapeutic or prophylactic effects will, of course, be determined by the particular circumstances surrounding the case, including, for example, the specific route of administration and response of the individual patient, the condition being treated and the severity of the patient's symptoms. In general, the compounds of the invention are most desirably administered at a concentration that will generally afford effective results without causing any serious side effects.

The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### Examples

Structural Analysis of FGFR1(D2/D3) Mutants

In order to test whether FGFR1 mutagenesis follow the proposed structure conserving path, we analyse the resulting proteins by far-UV CD spectroscopy. The CD spectra of 5 μM FGFR1(D2/D3) and FGFR1(D2/D3) mutants PA1101, PA1102, PA1103 and PA1104 in PBS (pH 7.4) are recorded on a Jasco J-710 spectropolarimeter (Japan Spectroscopic, Tokyo, Japan). Far-UV CD measurements are carried out in the range between 195-250 nm using a quartz cuvette with a path length of 0.1 cm, a response time of 0.25 s, and a data point resolution of 0.1 nm. Three scans are recorded to obtain smooth spectra. Mean residue ellipticities of background-corrected spectra are calculated by JASCO standard analysis. Analysis of the spectra with respect to secondary structure content is performed using the algorithm SELCON.

The secondary structure of the mutant protein is expected to closely resemble the structure of the wild type.

Investigating the Affinity of FGFR1 Mutants

For calculating the dissociation constant, Kd value, for the FGFR1(D2) wild type and mutant interactions with GAGs, surface Plasmon resonance (SPR) experiments were performed. The immobilisation of the biotinylated heparin onto a streptavidin coated C1 sensor chip was performed according to an established protocol. The actual binding interactions were recorded at 25° C. in PBS pH 7.4 containing 0.01% (v/v) P20 surfactant (BIAcore AB). 4 min injections of different protein concentrations at a flow rate of 30 μl/min were followed by 10 min dissociation periods in buffer and a pulse of 1 M sodium chloride for complete regeneration. The maximum response signals of protein binding to the heparin surface, corresponding to the plateaus of the respective sensorgrams, were used for Scatchard plot analysis and the calculation of equilibrium dissociation constants (Kd values). The experiments were all carried out at a protein concentration of 600 nM, which has been chosen on the basis of a series of ligand binding experiments performed at several protein concentrations ranging from 50 nM up to 1.2 μM.

By this means, Kd values of 465 nM for the wtFGFR1(D2) and 105 nM, 72.5 nM, 86.7 nM, and 63.2 nM were obtained for the mutants PA1101, PA1102, PA1103, and PA1104 respectively.

Inhibition of FGF2-Induced Proliferation by FGFR1 Mutants

Swiss albino murine fibroblasts (3T3 cells) are grown to confluency and then deprived of serum (calf bovine serum, CBS) from originally 10% to 1%. This leads to a general slow down of cell growth and hence to lesser confluency of adherent cells. Proliferation is induced by incubation with human FGF2 which could be inhibited by the addition of heparin but not by the addition of wtFGFR1. Adding the FGFR1(D2/D3) mutants PA1101-1104 to the FGF2-stimulated cells give almost the same reduction in proliferation, as measured by the %-age confluency, as the addition of heparin thereby clearly referring to the inhibitory activity of this mutant.

FGFR1 Mutants HUVEC Cell Sprouting Data

Method

HUVEC Cell Sprouting Assay (Angiogenesis)

The HUVEC sprouting assay was performed by ProQinase GmbH (Freiberg, Germany) based on a standard protocol. Briefly, HUVEC-derived spheroids were prepared by pipetting 500 endothelial cells (EC) in a hanging drop on plastic dishes to allow overnight spheroid aggregation. Fifty EC spheroids were then seeded in 0.9 ml of a collagen solution together with the appropriate concentration of the test compound and pipetted into individual wells of a 24-well plate to allow polymerisation. VEGF-A (25 ng/ml) or FGF-2 (25 ng/ml) in basal medium was added after 30 min by pipetting 100 μl of a 10-fold concentrated working dilution on top of the polymerized gel. The plates were incubated at 37° C. for 24 hours and fixed by adding 4% paraformaldehyde. Images from 10 spheroids per well/data point were randomly taken using an inverted microscope. The EC sprouts with branches of each spheroid were traced manually with the digital imaging software Analysis 3.2 (Soft imaging system, Münster, Germany). All measured lengths were added to give the cumulative sprout length (CSL) of this spheroid. The mean/median of the CSL of the 10 randomly selected spheroids is given as an individual data point.

Results

FGFR1 Mutants PA1102 and PA1103 Tested in the HUVEC Cell Sprouting Assay

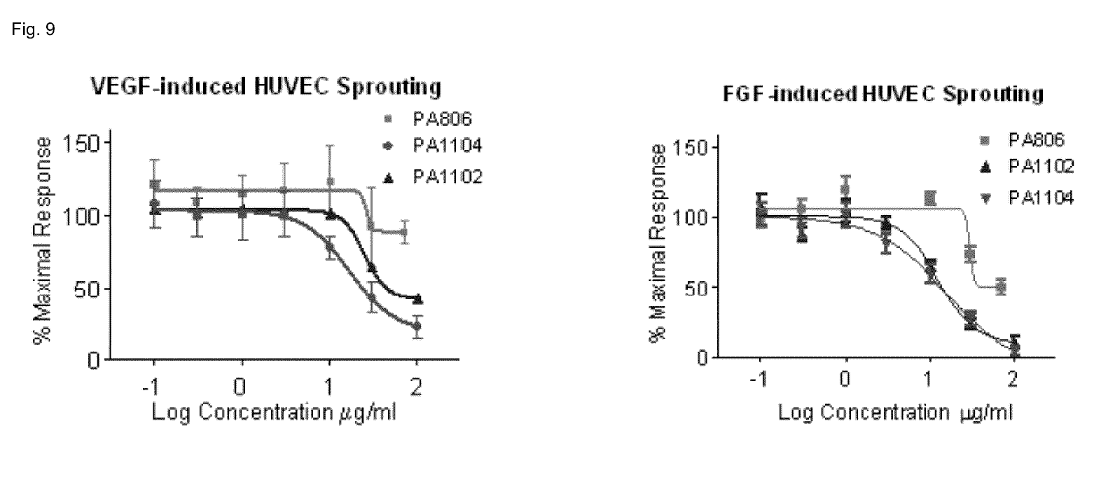

The FGFR1 Materials wT-PA806, PA1102 and PA1104 were likewise evaluated for their respective abilities to inhibit VEGF or FGF2-induced HUVEC cell sprouting. These materials also demonstrated a dose response with IC50 values>10 μg/mL (PA1102 and PA1104). Data is summarized in FIG. 9. No substantial differences were observed when comparing PA1102 and PA1104. However, test material PA806 (representing the wildtype control) showed only modest inhibition and only at the highest concentrations tested, whereas PA1102 and PA1104 show significant inhibition. Calculated IC50 values and related data are summarized in Table 1

TABLE 1
FGFR1 mutants testing in HUVEC sprouting assay: IC50 values.

| | Spheroid-based cellular angiogenesis assay | | | |
|---|---|---|---|---|
| | VEGF-A induced | | FGF2 induced | |
| | IC₅₀ (g/mL) | | IC₅₀ (g/mL) | |
| Test | 95% CI (g/mL) | | 95% CI (g/mL) | |
| article | Hill slope | r² | Hill slope | r² |
| wT-PA806 | >7.0 × 10⁻⁵ | | 7.1 × 10⁻⁵ | |
| | — | | 2.7 × 10⁻⁵-1.8 × 10⁻⁴ | |
| | — | — | −3.0 | 0.30 |
| PA1102 | 6.0 × 10⁻⁵ | | 1.5 × 10⁻⁵ | |
| | 3.6 × 10⁻⁵-1.0 × 10⁻⁴ | | 9.2 × 10⁻⁵-2.4 × 10⁻⁵ | |
| | −1.4 | 0.91 | −1.9 | 0.95 |
| PA1104 | 3.5 × 10⁻⁵ | | 1.6 × 10⁻⁵ | |
| | −1.6 × 10⁻⁵-7.9 × 10⁻⁵ | | 1.1 × 10⁻⁵-2.3 × 10⁻⁴ | |
| | −1.2 | 0.88 | −1.0 | 0.98 |
| Sutinib | 3.4 × 10⁻⁸ M | | 2.2 × 10⁻⁷ M | |
| | 1.4 × 10⁻⁸-8.2 × 10⁻⁸ M | | 6.6 × 10⁻⁸-7.4 × ⁻⁷ M | |
| | −2.0 | 0.86 | −1.2 | 0.87 |

This data was generated in the context of a research study valuating the efficacy of these materials in oncology related in vivo and in vitro assays. The studies demonstrate efficacy of FGFR1 mutants PA1102 and PA1104 to inhibit HUVEC sprouting induced by FGF2 and to a lesser extent VEGF.

1. A soluble FGFR1 mutant protein with increased GAG binding affinity compared to wild type FGFR1 protein, wherein the GAG binding region of said protein is modified by insertion of at least one basic amino acid and/or replacement of at least one non-basic amino acid by at least one basic amino acid.

2. The FGFR1 mutant protein of claim 1, wherein at least two non-basic amino acids in the GAG binding region are replaced by at least two basic amino acids.

3. The FGFR1 mutant protein of claim 1, wherein one or more of amino acid positions 210, 216 and/or 218 according to the numbering of SEQ ID NO:1 are modified.

4. The FGFR1 mutant protein of claim 1, wherein the basic amino acid is selected from the group consisting of lysine, arginine or histidine.

5. The FGFR1 mutant protein of claim 1, wherein said protein comprises the D2 or D2/D3 domain of FGFR1 or a functionally active part thereof.

6. The FGFR1 mutant protein of claim 1, wherein said protein consists of the D2 or of the D2/D3 domain of FGFR1 or a functionally active part thereof.

7. The FGFR1 mutant protein of claim 1, wherein said amino acid replacement and/or insertion is in the C-terminal region of the D2 domain, preferably between amino acids positions 206 and 248 of SEQ ID NO:1.

8. The FGFR1 mutant protein of claim 1, comprising the amino acid sequence:
TKPNRMP VAPY WTSPE(X1)ₙME(X2)ₘK LHAVPAA(X3)ₚTV
(X4)_{q}F(X5)ᵣCPSSGTP NPTLRWLKNG KEFKPDHRIGGYKVR(X6)ₛ
ATWS I(X7)ₜM(X8)ᵤSVVPSD KGNYTCIVEN EYGSINHTYQ
LDVV.
wherein:
X1, X2, X3, X4 and X5, consist of the wild type amino acid lysine or any of histidine or arginine;
X6 is the wild type amino acid tyrosine or any one of histidine, lysine, or arginine;
X7 is the wild type amino acid isoleucine or any one of histidine, lysine, or arginine; and
X8 is the wild type amino acid aspartate or any one of histidine, lysine, or arginine,
wherein any one of m, n, p, q, r, s, t, u is either 1 or 2, with the proviso that if m, n, p, q, r, s, t, u is 2, either of X1-X8 may be the wild type amino acid and/or any of the alternative amino acids,
and wherein at least two amino acid residues of the wild type sequence SEQ ID NO:1 are replaced by histidine, lysine or arginine, and optionally
wherein up to 2, 3, 4, or more than 4 additional amino acids are altered in a structure-conserving way.

9. The FGFR1 mutant protein of claim 1, wherein the amino acid sequence of the modified FGFR1 molecule is described by the following formula:
TKPNRMPVAPY WTSPE(X1)ₙME(X2)ₘK LHAVPAA(X3)ₚTV
(X4)_{q}F(X5)ᵣCPSSGTP NPTLRWLKNG KEFKPDHRIGGYKVR(X6)ₛ
ATWS I(X7)ₜM(X8)ᵤSVVPSD KGNYTCIVEN EYGSINHTYQ
LDVVERSPHR PILQAGLPAN KTVALGSNVE FMCKVYSDPQ
PHIQWLKHIE VNGSKIGPDN LPYVQILKTA GVNTTDKEME
VLHLRNVSFE DAGEYTCLAG NSIGLSHHSAWLTVLEALEE,
wherein:
X1, X2, X3, X4 and X5, consist of the wild type amino acid lysine or any of histidine or arginine;
X6 is the wild type amino acid tyrosine or any one of histidine, lysine, or arginine;
X7 is the wild type amino acid isoleucine or any one of histidine, lysine, or arginine; and
X8 is the wild type amino acid aspartate or any one of histidine, lysine, or arginine,
wherein any one of m, n, p, q, r, s, t, u is either 1 or 2, with the proviso that if m, n, p, q, r, s, t, u is 2, either of X1-X8 may be the wild type amino acid and/or any of the alternative amino acids,
and wherein at least two amino acid residues of the wild type sequence SEQ ID NO:1 are replaced by histidine, lysine or arginine and optionally wherein up to 2, 3, 4, or more than 4 additional amino acids are altered in a structure-conserving way.

10. The FGFR1 mutant protein of claim 8, wherein at least two amino acid residues of X1 to X8 of the wild type sequence are replaced by histidine, lysine or arginine.

11. The FGFR1 mutant protein of claim 10, wherein at least two amino acid residues of X6 to X8 of the wild type sequence are replaced by histidine, lysine or arginine.

12. The FGFR1 mutant protein of claim 1, further comprising a linker or marker peptide.

13. The FGFR1 mutant protein of claim 1, wherein the protein comprises the amino acid sequence of any one of SEQ ID NO: 4, 5, 6, and 7.

14. The FGFR1 mutant protein of claim 13, wherein the protein lacks the sequence HHHHHHSMG (SEQ ID NO:19).

15. (canceled)

16. A polynucleic acid molecule coding for a protein according to claim 1.

17. The polynucleic acid molecule of claim 16, wherein the molecule comprises a portion of a vector.

18. The polynucleic acid molecule of claim 17, wherein the vector is stably transfected into a recombinant cell, wherein the recombinant cell is not part of a human organism.

19. The FGFR1 mutant protein of claim 1, wherein the protein is formulated as a pharmaceutical composition comprising a pharmaceutically acceptable carrier.

20. A method of preventing or treating cancer by inhibiting or reducing angiogenesis during tumor growth or metastatic cancer, comprising the step of administering the FGFR1 mutant protein of claim 1 to a patient in need thereof.

21. The method of claim 20, wherein the patient suffers from a disease selected from the group consisting of breast cancer, lung cancer, bladder cancer, urothelial cell carcinoma (UCC), prostate cancer, myeloid and lymphoid neoplasms, myeloproliferative syndrome (EMS), rhabdomyosarcoma (RMS), atherosclerosis, brain cancer, esophageal carcinoma, chronic lymphocytic leukemia, hairy cell leukemia, multiple myeloma (MM), non-Hodgkins lymphoma, renal cell carcinoma, pancreatic carcinoma, colorectal cancer, uveal melanoma, involvement in genetic disorders, depression, bipolar disorder, schizophrenia, Alzheimer's disease and Huntington's disease.


SEQUENCE LISTING (20 sequences)

SEQ ID NO: 1 - protein, 822 aa, human [sequence omitted]

SEQ ID NO: 2 - protein, 100 aa, artificial
  FGFR1 with affinity tag
HHHHHHSMGTKPNRMPVAPYWTSPEKMEKKLHAVPAAKTVKFKCPSSGTPNPTLRWLKNGKEFKPDHRIGGYKVRYATWSIIMDSVVPSDKGNYTCIVEN

SEQ ID NO: 3 - protein, 116 aa, human
MSHHHHHHSMGTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVV

SEQ ID NO: 4 - protein, 116 aa, artificial
  mutant FGFR1
MSHHHHHHSMGTKPNRMPVAPYWTSPEKMEKKLHAVPAAKTVKFKCPSSGTPNPTLRWLKNGKEFKPDHRIGGYKVRKATWSIKMDSVVPSDKGNYTCIVENEYGSINHTYQLDVV

SEQ ID NO: 5 - protein, 116 aa, artificial
  mutant FGFR1
MSHHHHHHSMGTKPNRMPVAPYWTSPEKMEKKLHAVPAAKTVKFKCPSSGTPNPTLRWLKNGKEFKPDHRIGGYKVRKATWSIIMKSVVPSDKGNYTCIVENEYGSINHTYQLDVV

SEQ ID NO: 6 - protein, 116 aa, artificial
  mutant FGFR1
MSHHHHHHSMGTKPNRMPVAPYWTSPEKMEKKLHAVPAAKTVKFKCPSSGTPNPTLRWLKNGKEFKPDHRIGGYKVRYATWSIKMKSVVPSDKGNYTCIVENEYGSINHTYQLDVV

SEQ ID NO: 7 - protein, 116 aa, artificial
  mutant FGFR1
MSHHHHHHSMGTKPNRMPVAPYWTSPEKMEKKLHAVPAAKTVKFKCPSSGTPNPTLRWLKNGKEFKPDHRIGGYKVRKATWSIKMKSVVPSDKGNYTCIVENEYGSINHTYQLDVV

SEQ ID NO: 8 - DNA, 4746 bp, artificial
  pHAT2/FGFR1 mutant
taatacgactcactatagggaattgtgagcggataacaattcctctagaaataattttgtttaactttaagaaggagatatatccatgagtcatcaccatcaccatcactccatggggaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtgtaactcgagggatccgggccctctagatgcggccgcatgcataagcttgagtattctatagtgtcacctaaatcccagcttgatccggctgctaacaaagcccgaaaggaagctgagttggctgctgccaccgctgagcaataactagcataaccccttggggcctctaaacgggtcttgaggggttttttgctgaaaggaggaactatatccggataacctggcgtaatagcgaagaggcccgcaccgatcgcccttcccaacagttgcgcagcctgaatggcgaatggaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacagcttgcatgcctgcaggtcggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcgggtaccgagctcgaatttacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgat

SEQ ID NO: 9 - DNA, 4746 bp, artificial
  pHAT2/FGFR1 mutant
taatacgactcactatagggaattgtgagcggataacaattcctctagaaataattttgtttaactttaagaaggagatatatccatgagtcatcaccatcaccatcactccatggggaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgtaaagccacctggagcatcaaaatggactctgtggtgccctctgacaagggcaactacacctgcattgtggagaatgagtacggcagcatcaaccacacataccagctggatgtcgtgtaactcgagggatccgggccctctagatgcggccgcatgcataagcttgagtattctatagtgtcacctaaatcccagcttgatccggctgctaacaaagcccgaaaggaagctgagttggctgctgccaccgctgagcaataactagcataaccccttggggcctctaaacgggtcttgaggggttttttgctgaaaggaggaactatatccggataacctggcgtaatagcgaagaggcccgcaccgatcgcccttcccaacagttgcgcagcctgaatggcgaatggaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacagcttgcatgcctgcaggtcggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcgggtaccgagctcgaatttacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgat

SEQ ID NO: 10 - DNA, 4746 bp, artificial
  pHAT2/FGFR1 mutant
taatacgactcactatagggaattgtgagcggataacaattcctctagaaataattttgtttaactttaagaaggagatatatccatgagtcatcaccatcaccatcactccatggggaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgtaaagccacctggagcatcataatgaaatctgtggtgccctctgacaagggcaactacacctgcattgtggagaatgagtacggcagcatcaaccacacataccagctggatgtcgtgtaactcgagggatccgggccctctagatgcggccgcatgcataagcttgagtattctatagtgtcacctaaatcccagcttgatccggctgctaacaaagcccgaaaggaagctgagttggctgctgccaccgctgagcaataactagcataaccccttggggcctctaaacgggtcttgaggggttttttgctgaaaggaggaactatatccggataacctggcgtaatagcgaagaggcccgcaccgatcgcccttcccaacagttgcgcagcctgaatggcgaatggaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacagcttgcatgcctgcaggtcggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcgggtaccgagctcgaatttacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgat

SEQ ID NO: 11 - DNA, 4746 bp, artificial
  pHAT2/mutant FGFR 1
taatacgactcactatagggaattgtgagcggataacaattcctctagaaataattttgtttaactttaagaaggagatatatccatgagtcatcaccatcaccatcactccatggggaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcaaaatgaaatctgtggtgccctctgacaagggcaactacacctgcattgtggagaatgagtacggcagcatcaaccacacataccagctggatgtcgtgtaactcgagggatccgggccctctagatgcggccgcatgcataagcttgagtattctatagtgtcacctaaatcccagcttgatccggctgctaacaaagcccgaaaggaagctgagttggctgctgccaccgctgagcaataactagcataaccccttggggcctctaaacgggtcttgaggggttttttgctgaaaggaggaactatatccggataacctggcgtaatagcgaagaggcccgcaccgatcgcccttcccaacagttgcgcagcctgaatggcgaatggaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacagcttgcatgcctgcaggtcggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcgggtaccgagctcgaatttacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgat

SEQ ID NO: 12 - DNA, 4746 bp, artificial
  pHAT2/mutant FGFR1
taatacgactcactatagggaattgtgagcggataacaattcctctagaaataattttgtttaactttaagaaggagatatatccatgagtcatcaccatcaccatcactccatggggaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgtaaagccacctggagcatcaaaatgaaatctgtggtgccctctgacaagggcaactacacctgcattgtggagaatgagtacggcagcatcaaccacacataccagctggatgtcgtgtaactcgagggatccgggccctctagatgcggccgcatgcataagcttgagtattctatagtgtcacctaaatcccagcttgatccggctgctaacaaagcccgaaaggaagctgagttggctgctgccaccgctgagcaataactagcataaccccttggggcctctaaacgggtcttgaggggttttttgctgaaaggaggaactatatccggataacctggcgtaatagcgaagaggcccgcaccgatcgcccttcccaacagttgcgcagcctgaatggcgaatggaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacagcttgcatgcctgcaggtcggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcgggtaccgagctcgaatttacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgat

SEQ ID NO: 13 - protein, 105 aa, artificial
  mutant FGFR1
TKPNRMPVAPYWTSPEXMEXKLHAVPAAXTVXFXCPSSGTPNPTLRWLKNGKEFKPDHRIGGYKVRXATWSIXMXSVVPSDKGNYTCIVENEYGSINHTYQLDVV

SEQ ID NO: 14 - protein, 221 aa, artificial
  mutant FGFR1
TKPNRMPVAPYWTSPEXMEXKLHAVPAAXTVXFXCPSSGTPNPTLRWLKNGKEFKPDHRIGGYKVRXATWSIXMXSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEE

SEQ ID NO: 15 - DNA, 351 bp, artificial
  mutant FGFR1
atgagtcatcaccatcaccatcactccatggggaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgtaaagccacctggagcatcaaaatggactctgtggtgccctctgacaagggcaactacacctgcattgtggagaatgagtacggcagcatcaaccacacataccagctggatgtcgtgtaa

SEQ ID NO: 16 - DNA, 351 bp, artificial
  mutant FGFR1
atgagtcatcaccatcaccatcactccatggggaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgtaaagccacctggagcatcataatgaaatctgtggtgccctctgacaagggcaactacacctgcattgtggagaatgagtacggcagcatcaaccacacataccagctggatgtcgtgtaa

SEQ ID NO: 17 - DNA, 351 bp, artificial
  mutant FGFR1
atgagtcatcaccatcaccatcactccatggggaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcaaaatgaaatctgtggtgccctctgacaagggcaactacacctgcattgtggagaatgagtacggcagcatcaaccacacataccagctggatgtcgtgtaa

SEQ ID NO: 18 - DNA, 351 bp, artificial
  mutant FGFR1
atgagtcatcaccatcaccatcactccatggggaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgtaaagccacctggagcatcaaaatgaaatctgtggtgccctctgacaagggcaactacacctgcattgtggagaatgagtacggcagcatcaaccacacataccagctggatgtcgtgtaa

SEQ ID NO: 19 - protein, 9 aa, artificial
  leader sequence
HHHHHHSMG

SEQ ID NO: 20 - DNA, 11 bp, artificial
  signal sequence
mshhhhhhsmg